(19) United States
(12) Patent Application Publication
(10) Pub. No.: US 2004/0096418 A1
(43) Pub. Date: May 20, 2004

(54) Cosmetically effective composition containing malva sylvestris and mentha piperita extracts

(76) Inventors: Thomas Gafner, Biel-Benken (CH); Thomas Schreier, Bubendorf (CH); Dominik Imfeld, Basel (CH)

Correspondence Address: JONES DAY, 222 EAST 41ST STREET, NEW YORK, NY, 10017, US

(21) Appl. No.: 10/468,419

(22) PCT Filed: Feb. 16, 2001
(86) PCT No.: PCT/CH01/00103

(51) Int. Cl. A61K 7/06; A61K 35/78

(52) U.S. Cl. 424/74; 424/747; 424/725

(57) ABSTRACT

The invention relates to a concentrate for the production of cosmetically effective compositions that lighten the skin, wherein said concentrate contains at least one extract (i) of mallow (malva sylvestris) and (ii) peppermint (mentha piperita) and optionally an additional extract selected from amongst the extracts of (iii) cowslip (primula veris), (iv) lesser burnet (pimpinella saxifraga), (v) common speedwell (veronica officinalis), (vi) lemon balm (melissa officinalis) and (vii) common yarrow (achillea millefolium).

The present invention refers to a concentrate that is suitable for manufacturing compositions that can be used to effectively whiten the skin. The concentrate comprises at least one mallow plant extract (Malva sylvestris) and at least one peppermint extract (Mentha piperita). The concentrate according to the present invention can contain further plant extracts. The extracts are taken preferably from the root, the stem, leaves and the flower.

The human epidermis is constantly exposed to irritants such as UV-radiation, fluctuating temperatures and humidity. It was now found that cosmetic products that contain the extracts according to the present invention not only have a soothing and moisturising effect on the skin but in particular also whiten the skin. Thus, the concentrate according to the present invention is used as a skin-whitening cosmetic ingredient.

The present invention is defined in the claims and refers in particular to a concentrate that is used to manufacture cosmetic compositions that effectively whiten the skin and are characterized in that said concentrate comprises (i) at least one mallow plant extract (malva sylvestris) and (ii) at least one peppermint extract (mentha piperita) in a cosmetically effective concentration.

Preferably, the concentrate contains at least one other extract that is selected from the following extracts: (iii) primrose (primula veris), (iv) pimpernell (pimpinella saxifraga), (v) speedwell (veronica officinalis), (vi) lemon balm (melissa officinalis), and (vii) milfoil (achillea millefolium).

Preferably, the concentrate comprises at least one extract each from (i), (ii) and (iii), preferably at least one extract each from (i), (ii), (iii) and (iv), preferably at least one extract each from (i), (ii), (iii), (iv), (v) and (vi), preferably one extract each from (i), (ii), (iii), (iv), (v), (vi) and (vii).

The present invention also refers to the use of the concentrate according to the present invention for the manufacture of cosmetically effective compositions.

Furthermore, the present invention refers to a cosmetically effective composition resp. formulation for cosmetic application which is characterised in that said concentrate comprises at least one extract of the (i) mallow plant (malva sylvestris) and at least (ii) one peppermint extract (mentha piperita) in a cosmetically effective concentration.

Preferably, the concentrate comprises at least one other extract that is derived from the following extracts: (iii) primrose (primula veris), (iv) pimpernell (pimpinella saxifraga), (v) speedwell (veronica officinalis), (vi) lemon balm (melissa officinalis), and (vii) milfoil (achillea millefolium).

The present invention also refers to a process for manufacturing a concentrate that comprises the aforementioned extracts that are suitable for the manufacture of the cosmetically effective compositions, characterized in that each time the dried plant (root, stem, leaves and/or flower) is extracted with a suitable solvent, preferably with water and/or a suitable organic solvent, preferably with a suitable alcohol or a suitable ketone or a mixture of these compounds, preferably with water and ethanol, resp. a mixture of water and ethanol, the resulting suspension being then filtered, the organic solvents removed, and the suspension then evaporated to a concentrate.

The plants (i) mallow (malva sylvestris), (ii) peppermint (mentha piperita) (iii) primrose (primula veris), (iv) pimpernell (pimpinella saxifraga), (v) speedwell (veronica officinalis), (vi) lemon balm (melissa officinalis) and (vii) milfoil (achillea millefolium) are well-known medicinal plants that are described in pharmaceutical publications (pharmacopoeia).

A preferred method for manufacturing the extracts is to extract the plants (root, stem, leaves and/or flower) in a first step with water, preferably at a temperature within the range of 50° C. to 100° C., preferably at about 90° C. and at a plant material/water weight ratio of 1:1 to 1:100, preferably of about 1:10. In other words, one should use for example 10 kg of water per 1 kg of dried plant material. After filtration, the wet residue is extracted with a suitable organic solvent or a mixture of such solvents, for example ethanol and/or acetone, optionally mixed with water at a weight ratio of organic solvent to water of 2:8 to 1:1, preferably with ethanol (approx. 95%), at a weight ratio of dried plant material to solvent ranging from 1:1 to 1:100, preferably from 1:1 to 1:10, preferably within a weight ratio of about 1:3. Extraction lasts for example about 1 to 100 hours, preferably about 20 hours at room temperature while being stirred. Extraction may be repeated under the same conditions between 10 minutes and 10 hours, preferably for about 2 hours. It is also possible to extract exclusively with organic solvents.

The unified filtrates are then concentrated to a concentrate using distillation and are thus freed from organic solvents at the same time. The concentrated filtrates are then filtered. The resulting concentrate is preferably a aqueous concentrate and may be formulated with glycerine or another compatible solvent such as propylene glycol, or a mixture of such solvents.

The weight ratios of the individual extracts preferably range from 1:10 to 10:1, preferably in the range from 1:4 to 4:1 and preferably at about 1:1.

The extract or concentrate manufactured in this way is usually an opaque fluid with a characteristic odour and has an acid value (pH) of about 5.0 to 7.0, preferably of about 6.0. The extract usually contains about 5% to 20% by weight of dry matter, preferably from 7% to 10% by weight, in relation to the total weight of the aqueous extract. This extract is preferably used in concentrations of 3% to 5% by weight, in relation to the total composition.

The concentrate according to the present invention can be further processed at room temperature or at a slightly increased temperature, for example at about 40° C. to 80° C. Preferably, the extract is added to the aqueous phase of the cosmetic formulation.

The cosmetically effective composition, also referred to as the formulation or the preparation, which contains the concentrate may be present resp. may be prepared in any way desired for application. Thus these formulations can be for example either an aqueous preparation, an emulsion or micro-emulsion of the type water-in-oil (w/o) or oil-in-water (o/w), a multiple emulsion, for example, of the type water-in-oil-in-water (w/o/w), a gel, a solid or an aerosol. Said formulations may be prepared to represent for example a powder, a wet compress, a lotion, a creme or any other cosmetically approved form. Lotions and cremes are the preferred forms. Gels are used less frequently for skin whiteners.

Preferably, the applied concentrations of the concentrate (based on dry matter) is approximately 0.1% to 10.0% by weight, preferably about 3.0 and 5.0% by weight, in relation to the total weight of the cosmetic composition. The production of such cosmetic compositions as well as production methods, basic raw materials and additives, are well known from the literature.

As a rule, these raw materials and additives can be used to produce the cosmetically effective composition or formulation as defines herein. Examples of such basic raw materials are for example water, vegetable, animal or synthetic and semi-synthetic materials such as moisturisers, alcohols, polyhydric alcohols, fatty acids, fats, oils, wax, thickeners, surface-active compounds, such as emulsifiers, fillers, antiseptic compounds, antioxidants, compounds that absorb UV-radiation and UV-quenchers, vitamins such as vitamin A, vitamin B and/or vitamin E, preservatives, bactericides, aromatics and perfumes as well as dyestuffs, foam stabilisers and/or silicones.

Raw materials and additives to be used in the present invention are in particular glycerine, polyglycerine compounds, ethylene glycol, propylene glycol, polyethylene glycol, polypropylene glycol, ethyl alcohol, isopropyl alcohol, agar gum, tragacanth gum, arabic gum, vegetable or animal gelatines, methyl cellulose, ethyl cellulose, carboxymethyl cellulose, hydroxy-methyl cellulose, hydroxypropyl cellulose, sodium alginate, polyvinyl alcohol, polyvinyl alcohol acetate esters, C₆₋₂₂-fatty alcohols, such as cetyl alcohol, C₆₋₂₂-fatty acid ester, in particular stearic acid, palmitic acid, lauric acid and the related methyl, ethyl and propyl esters, lanolin, liquid paraffin or natural or synthetic wax, such as vaseline or bees wax, vegetable oils such as olive oil, coconut oil, soy-bean oil, castor oil, and suitable saturated oils, hydroxyl containing compounds modified with polyalkylene oxides, as well as other known raw materials used in the production of cosmetic formulations.

The whitening effect of skin-whitening substances depends greatly on how said substances are able to penetrate the epidermis to the basal cell layer, because the melanocytes in which the skin pigment melanin is formed are located in the basal cell layer. For that reason, the effect of the substance is directly dependent on the composition of the formulation. To improve penetration of the substance, the formulation preferably contains so-called penetration accelerators which weaken the epidermal permeability barrier and thus ease the penetration of the stratum corneum. Penetration accelerators have already been mentioned and are well-known. Preferred penetration accelerators are, for example, unsaturated fatty acids such as oleic acid, unsaturated oleic acids, linolic acid, but also compounds such as alcohol glycol, alcoxypolyglycol, such as ethoxy diglycol, azon derivatives and other penetration accelerators known from the literature.

In order to manufacture an emulsion or micro-emulsion of the type water-in-oil (w/o) or oil-in-water (o/w), or water-in-oil-in-water (w/o/w), it is best to use compounds known to be used for this purpose and known methods. For the manufacture of the lipid phase, preferably mineral or natural oils or waxes are used. Synthetic esters from fatty acids with alcohols may also be used, such as ester from fatty acids with ethanol, propanol, isopropanol, propylene glycol or glycerine, or esters of fatty alcohols with organic C₃₋₂₀-acids. Preferred substances are esters of myristic acid, palmitic acid, stearic acid, oleic acid, such as propyl myristate, isopropyl palmitate, isopropyl stearate, isopropyl oleate, butyl stearate, hexyl laurate, 2-hexyldecyl stearate, or natural oils such as jojoba oil or a mixture of such compounds. Preferred types of silicones are in particular dimethylpolysiloxanes, preferably in cyclic or linear form.

The aqueous phase of the formulations according to the present invention may contain numerous water-soluble or emulsifiable compounds as is known to the expert in the art. Such compounds are, for example, natural or synthetic compounds, such as alcohols, polyhydric alcohols and/or ethers, as already mentioned. Other agents and additives for aqueous or oily cosmetic formulations are, for example, fillers such as titanium oxide, zinc oxide, magnesium carbonate, talcum, kaolin, silica, preferably in colloidal form, or starch.

Furthermore, the present formulations can contain acids or bases for setting the acid value (pH-value), such as sodium hydroxide, phosphoric acid or lactic acid, triethanolamine, preferably in a buffer system form.

A large number of compounds that are suitable for the purposes mention above are known. The expert in the art knows how to optimise the use of these compounds.

The invented concentrate can be stored without damage at a temperature of 4° C. to 8° C. for over one year in airtight containers that are protected from light.

The following example illustrate the invention.

### EXAMPLE 1

### Manufacture of the Concentrate

10 parts of dried plant material, containing one part of each plant: (i) mallow plant (malva sylvestris), (ii) peppermint (mentha piperita), (iii) primrose (primula veris), (iv) pimpernell (pimpinella saxifraga), (v) speedwell (veronica officinalis), (vi) lemon balm (melissa officinalis) and (vii) milfoil (achillea millefolium) were finely cut and subsequently extracted twice with 100 parts of an aqueous ethanol solution containing 30 parts of water and 70 parts of ethanol. The resulting suspension was filtered, and the filtrate was concentrated under reduced pressure to a concentrate, whereby the alcohol was distilled off.

The resulting concentrate was formulated with glycerine by adding the same amount of glycerine into the concentrate while stirring. No pathogenic germs were found. All relevant safety tests used in the cosmetic industry were fulfilled. The test results also gave no indication to any environmental risks. It is possible to add to the concentrate same amounts of glycerine or a compatible solvent or optionally even higher amounts.

The concentrate can be further processed to a formulation in a manner known per se for example by adding other compatible solvents and additives as described earlier in the text. This is the way the formulation was manufactured listed in detail in Table 1. The concentrate as well as the cosmetic product made from it have a soothing effect on sensitive skin and exhibit a noticeable skin-whitening effect.

TABLE 1

| | Ingredients | INCI name | Weight in % |
|---|---|---|---|
| 1 | Brij 721 | Steareth-21 | 5.6 |
| 2 | Brij 72 | Steareth2 | 1.9 |
| 3 | cetyl alcohol | cetyl alcohol | 1 |
| 4 | oleic acid | oleic acid | 5 |
| 5 | paraffin oil | mineral oil | 5 |
| 6 | isopropyl | isopropyl myristate | 5 |
| | myristate | | |
| 7 | Trivalin SF | ethoxy diglycol | 5 |
| 8 | Parsol 1789 | (UV-A filter) | 3 |
| 9 | Parsol MCX | (UV-B filter) | 5 |
| 10 | Phenonip | | 0.5 |
| 11 | propylene Glycol | propylene Glycol | 5 |
| 12 | plant extract | (skin-whitening substance as | 5 |
| | with skin- | described in example 1) | |
| | whitening | | |
| | component | | |
| 13 | water | | q.s 100 |

1. A concentrate that is suitable for the manufacturing of cosmetically active compositions that whiten the skin, characterised in that said concentrate comprises at least one (i) mallow plant extract (malva sylvestris) and at least (ii) one peppermint extract (mentha piperita) in an cosmetically effective concentration.

2. A concentrate according to claim 1, characterised in that said concentrate contains at least one other extract selected from the following extracts: (iii) primrose (primula veris), (iv) pimpernell (pimpinella saxifraga), (v) speedwell (veronica officinalis), (vi) lemon balm (melissa officinalis), and (vii) milfoil (achillea millefolium).

3. A concentrate according to claims 1 or 2, characterised in that said concentrate contains at least one extract each from (i), (ii) and (iii), preferably at least one extract each from (i), (ii), (iii) and (iv), preferably at least one extract each from (i), (ii), (iii), (iv), (v) and (vi), preferably one extract each from (i), (ii), (iii), (iv), (v), (vi) and (vii).

4. A concentrate according to any one of the claims 1-3, characterised in that the weight ratios of the individual extracts to each other range from 1:10 to 10:1, preferably from 1:4 to 4:1, and preferably are at about 1:1.

5. A concentrate according to any one of the claims 1-4, characterised in that said concentrate has an acid value of 5.0 to 7.0, preferably of about 6.0.

6. A concentrate according to any one of the claims 1-5, characterised in that said concentrate contains 5% to 20% by weight, preferably 7% to 10% by weight of dry matter, in relation to the total weight of the aqueous extract.

7. A concentrate according to any one of the claims 1-6, characterised in that it is a aqueous concentrate which is formulated with glycerine and/or propylene glycol.

8. Use of the concentrate according to any one of the claims 1-7, for the manufacture of cosmetically active compositions.

9. Cosmetically active composition, characterised in that said composition comprises a concentrate according to any one of the claims 1-6.

10. Cosmetically active composition according to claim 9, characterised in that said composition is an aqueous preparation, being an emulsion or micro-emulsion of the type water-in-oil (w/o) or oil-in-water (o/w), a multiple emulsion, a gel, in a solid form or an aerosol.

11. Cosmetically active composition according to claim 10, characterised in that said composition is present in the form of a powder, a wet compress, a lotion, a creme or is present in any other permissible cosmetic form, preferably in the form of a lotion or as a creme.

12. Cosmetically active composition according to any one of the claims 1-11, characterised in that said composition comprises a concentrate (based on the concentrate-dry matter) according to any one of the claims 1-6, in a concentration of 0.1 to 10.0% by weight, preferably 3.0 to 5.0%, in relation to the total weight of the cosmetic composition.

13. Cosmetically active composition according to any one of the claims 9-12, characterised in that it contains a penetration accelerator which preferably is selected from unsaturated fatty acids, alkoxy glycols, alkoxy polyglycols and azon derivatives.

14. Process for manufacturing a concentrate according to any one of the claims 1-6, characterised in that the dried plant (root, stem, leaves and/or flower) is extracted with a suitable solvent, preferably with water and/or a suitable organic solvent, preferably alcohol or suitable ketone or a mixture of these compounds, preferably water and ethanol, or a mixture of water and ethanol, and optionally the resulting suspension being then filtered, freed of organic solvents and then concentrated.
